(19) United States
(12) Patent Application Publication
(10) Pub. No.: US 2025/0127538 A1
(43) Pub. Date: Apr. 24, 2025

(54) ADJUSTABLE FLEXIBLE CANNULA

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Rick Fu, Randolph, MA (US); Kendra O'Malley, Charlton, MA (US); Fabiano Paiva-Filho, Plainville, MA (US); Justin A. Barbas, West Bridgewater, MA (US)

(73) Assignee: Smith & Nephew, Inc., Memphis, TN (US)

(21) Appl. No.: 18/890,997

(22) Filed: Sep. 20, 2024

Related U.S. Application Data
(60) Provisional application No. 63/591,499, filed on Oct. 19, 2023.

(51) Int. Cl. A61B 17/34

(52) U.S. Cl. CPC A61B 17/3431; A61B 2017/3443

(57) ABSTRACT

Adjustable, flexible cannulas of this disclosure include a body having a proximal end and a distal end. The body defines a through hole extending from the proximal end to the distal end. The distal end of the body has a flange for engaging a surface of tissue. An outer surface of the body includes a plurality of axially spaced first engagement features extending around a circumference of the body. A moveable member has a lumen for receiving the body therethrough. An inner surface of the lumen has a plurality of second engagement features for selectively engaging a corresponding one of the first engagement features. The moveable member is moveable along a length of the body to adjust a distance between the flange and the moveable member.

### CROSS-REFERENCE TO RELATED APPLICATIONS

This application claims priority to and benefit of U.S. Provisional Application No. 63/591,499 filed Oct. 19, 2023, entitled ADJUSTABLE FLEXIBLE CANNULA, the contents of which are incorporated herein by reference in their entirety for all purposes.

### FIELD

The present disclosure relates generally to surgical cannulas and, more particularly, to length-adjustable, flexible cannulas.

### BACKGROUND

During arthroscopic surgical procedures, surgeons often use a cannula for uninterrupted access to a joint space in a patient's body. The cannula allows the surgeon to pass instruments, sutures, and grafts into the joint space, to avoid tissue bridges, and to control saline fluid loss. However, typical cannulas are long and rigid, making them difficult to pass through a surgical opening. Typical cannulas can also restrict mobility and limit access to areas within the body.

### SUMMARY

The disclosure describes an adjustable-length, flexible cannula that solves deficiencies in existing cannulas. The cannula has a main body and a moveable component disposed on the main body for adjusting a distance between the moveable component and a flange on the distal end of the cannula based on the physical characteristics of the patient. Once the moveable component has been adjusted to the correct position, the excess cannula body proximal to the moveable member can be trimmed. The distal flange is configured to bend, facilitating insertion and removal of the cannula from the joint space. Furthermore, an internal fluid management component creates a seal around an instrument that is inserted into the cannula, preventing back-flow of fluid from the patient's body through the cannula.

Embodiments of the adjustable, flexible cannula of this disclosure may include one or more of the following, in any suitable combination.

Embodiments of the cannula of this disclosure include a body having a proximal end and a distal end. The body defines a through hole extending from the proximal end to the distal end. The distal end of the body includes a flange for engaging a surface of tissue. An outer surface of the body has a plurality of axially spaced first engagement features extending around a circumference of the body. A moveable member has a lumen for receiving the body through the lumen. An inner surface of the lumen includes a plurality of second engagement features for selectively engaging a corresponding one of the first engagement features. The moveable member is moveable along a length of the body to adjust a distance between the flange and the moveable member.

In further embodiments, the moveable member is a flange. In embodiments, the moveable member includes a plurality of suture management features. In embodiments, the first engagement features are cylindrical barbs having a negative rake angle such that the face of the barb slopes away from the outer edge of the barb. In embodiments, the second engagement features are cylindrical channels. Each channel is configured to receive a corresponding one of the barbs. In embodiments, the plurality of second engagement features is at least three second engagement features. In embodiments, the plurality of second engagement features is five second engagement features. In embodiments, at least one of the body and the moveable member is made of a flexible material. In embodiments, the through hole includes an internal dam defining an opening therethrough. A diameter of the opening is selected to be smaller than a diameter of the through hole. In embodiments, a size of the opening is selected to allow passage of a surgical instrument while simultaneously forming a seal around the instrument. In embodiments, the cannula further includes a slit feature configured to allow for passage and removal of a surgical instrument while simultaneously forming a seal around the surgical instrument. In embodiments, the slit feature is located proximal of the flange. In embodiments, a cross section of the flange includes a plurality of webbed areas. A width of the webbed areas is selected to be thinner than a width of a surrounding area of the flange to allow the flange to bend along the webbed areas. In embodiments, opposing webbed areas of the flange define a hole through the webbed area. The hole is configured to engage an insertion instrument passed through the cannula.

Embodiments of a method of inserting a cannula into a body of a patient of this disclosure include placing a flange of the cannula into contact with an internal tissue the patient. The cannula also includes a body having a proximal end and a distal end. The body defines a through hole extending from the proximal end to the distal end. The distal end of the body includes the flange. An outer surface of the body has a plurality of axially spaced first engagement features extending around a circumference of the body. A moveable member has a lumen for receiving the body through the lumen. An inner surface of the lumen has a plurality of second engagement features for selectively engaging a corresponding one of the first engagement features. The method also includes moving the moveable member along a length of the body to adjust a distance between the flange and the moveable member. In embodiments, moving the moveable member includes placing the moveable member into contact with an outside skin of the patient. In embodiments, the method further includes trimming the body of the cannula distal to the moveable member.

Embodiments of a method of inserting a surgical instrument into a body of a patient of this disclosure include placing a flange of the cannula into contact with an internal tissue the patient. The cannula also includes a body having a proximal end and a distal end. The body defines a through hole extending from the proximal end to the distal end. The distal end of the body includes the flange. An outer surface of the body has a plurality of axially spaced first engagement features extending around a circumference of the body. A moveable member has a lumen for receiving the body through the lumen. An inner surface of the lumen has a plurality of second engagement features for selectively engaging a corresponding one of the first engagement features. The method also includes moving the moveable member along a length of the body to adjust a distance between the flange and the moveable member. The surgical instrument is then passed through the cannula into the body of the patient. In embodiments, the method further includes passing the surgical instrument through an opening of an internal dam of the cannula. A diameter of the opening is selected to be smaller than a diameter of the through hole. In embodiments, the method further includes passing the surgical instrument through a slit feature of the cannula. The slit feature is configured to allow for passage and removal of the surgical instrument while simultaneously forming a seal around the surgical instrument.

A reading of the following detailed description and a review of the associated drawings will make apparent these and other features and advantages. Both the foregoing general description and the following detailed description are explanatory only and are not restrictive of aspects as claimed.

### BRIEF DESCRIPTION OF THE DRAWINGS

The disclosure will be more fully understood by reference to the detailed description, in conjunction with the following figures, wherein:

FIGS. 1A and 1B illustrate an embodiment of an adjustable, flexible cannula of this disclosure;

FIGS. 2A-2C illustrate the movement of a moveable member along a length of the cannula, according to some embodiments;

FIGS. 3A-3C illustrate the use of the cannula, according to some embodiments;

FIGS. 4A and 4B illustrate features of the cannula that allow the surgeon to prevent back-flow of fluid from the body, according to some embodiments;

FIGS. 5A-5C illustrate features of a flexible flange at the distal end of the cannula, according to some embodiments;

FIGS. 6A and 6B illustrate an alternative embodiment of an adjustable, flexible cannula of this disclosure; and

FIGS. 7A and 7B illustrate another alternative embodiment of an adjustable, flexible cannula of this disclosure.

### DETAILED DESCRIPTION

In the description that follows, like components have been given the same reference numerals, regardless of whether they are shown in different embodiments. To illustrate embodiment(s) in a clear and concise manner, the drawings may not necessarily be to scale, and certain features may be shown in somewhat schematic form. Features that are described and/or illustrated with respect to one embodiment may be used in the same way or in a similar way in one or more other embodiments and/or in combination with or instead of the features of the other embodiments.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” and/or plural forms of each are open ended and include the listed parts and can include additional parts that are not listed. “And/or” is open-ended and includes one or more of the listed parts and combinations of the listed parts. Use of the relative terms “top,” “bottom,” “above,” “below” and the like helps only in the clear description of the disclosure and does not limit the structure, positioning and/or operation of the disclosure in any manner.

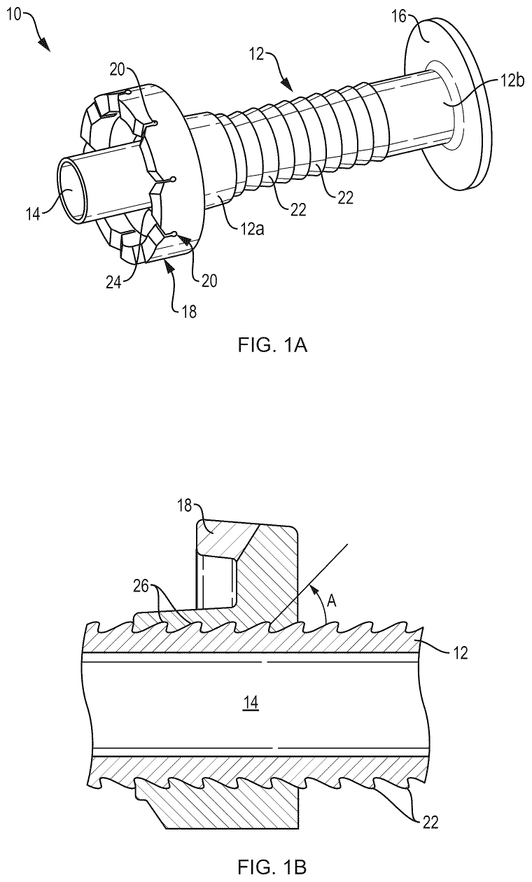

FIGS. 1A and 1B illustrate an embodiment of an adjustable, flexible cannula 10 of this disclosure for allowing the passage of, e.g., surgical instruments, grafts, and sutures from the outside of a patient's body into a joint space. Examples of surgical instruments include, but are not limited to, arthroscopes, forceps, graspers, drills, cutters, and anchor insertion devices. Other examples of surgical instruments include smaller cannulas for injecting healing compositions or removing bodily fluids from the joint space. The cannula 10 may allow for both improved visualization of the joint space and the stabilization of the cannula 10 in the patient's body.

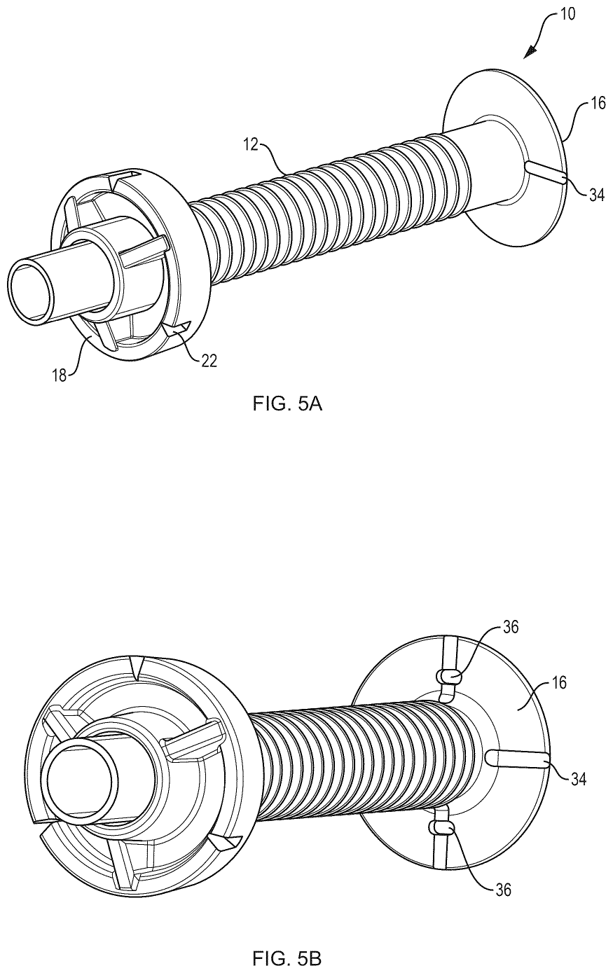

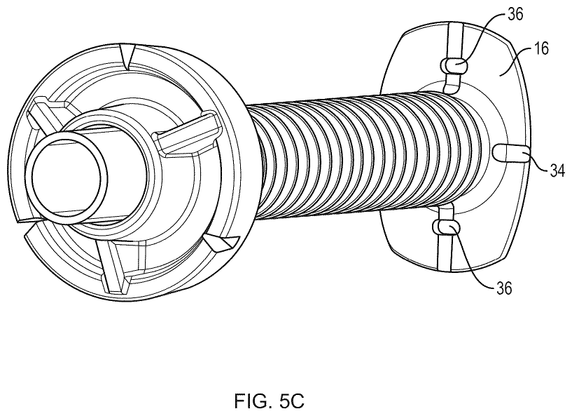

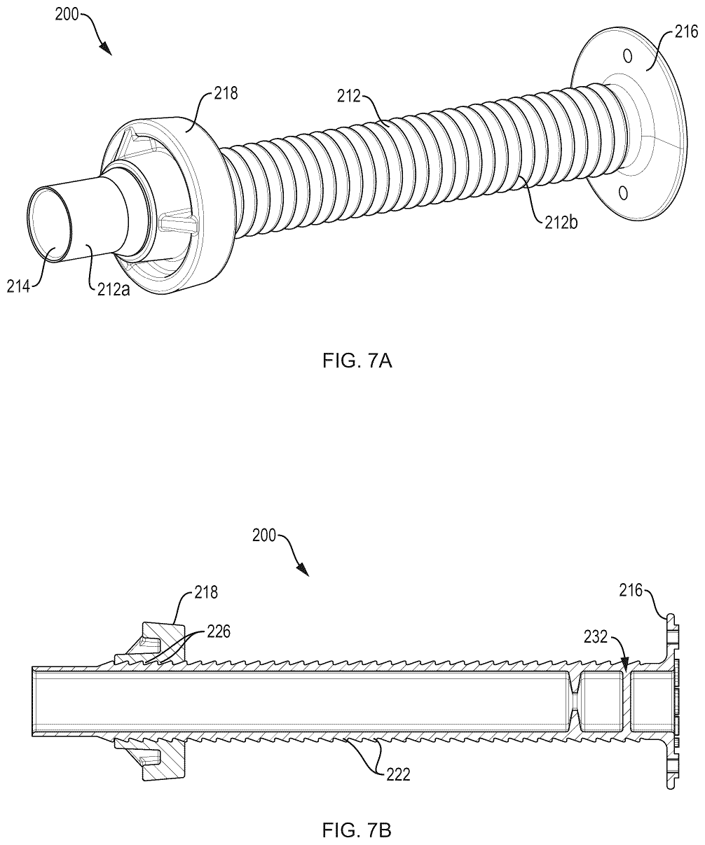

As shown in FIG. 1A, the cannula 10 may generally include a tubular body 12 having a through hole 14 extending from a proximal end 12a of the body 12 to a distal end 12b. A diameter of the through hole 14 may be selected to allow for the passage of surgical instruments, sutures, and/or grafts. For example, a diameter of the through hole 14 may be between 6 mm and 12 mm. An outer surface of the body 12 may include a plurality of axially spaced first engagement features 22 extending around a circumference of the body 12. A flexible flange 16 may be provided at the distal end 12b of the body 12. A moveable member 18, which may also be a flange, may be disposed about the body 12 towards the proximal end 12a of the body 12. The moveable member 18 may define a lumen 24 for receiving the body 12 therethrough. The moveable member 18 may also include a plurality of suture management features 20 for holding tensioned sutures in place as instruments, sutures, and grafts are passed through the cannula 10. For example, the suture management features 20 may be cleats, as shown. The cleats can have a variety of shapes, such as the keyhole shape shown in FIG. 1A, the rectangular shape shown in FIG. 5A, or the triangle shape shown in FIG. 5B. Alternatively, the moveable member 18 may lack suture management features, as shown in FIG. 7A.

As shown in FIG. 1B, an inner surface of the lumen 24 may comprise a plurality of second engagement features 26 for selectively engaging a corresponding number of the first engagement features 22 to tighten the interface between the moveable member 18 and the body 12. For example, the first engagement features 22 may be cylindrical barbs having a negative rake angle A such that the face of the barb slopes away from the outer edge of the barb. The second engagement features 26 may in turn be a plurality of cylindrical channels, each one configured to receive a corresponding one of the barbs. However, the disclosure contemplates other suitable engagement features 22, 26, such as threads, or round or square ribs. In embodiments, the number of second engagement features 26 may be at least three second engagement features 26 for receiving at least three first engagement features 22. In further embodiments, the number of corresponding first and second engagement features 22, 26 may be five, as shown. However, the disclosure also contemplates fewer than three corresponding first and second engagement features 22, 26.

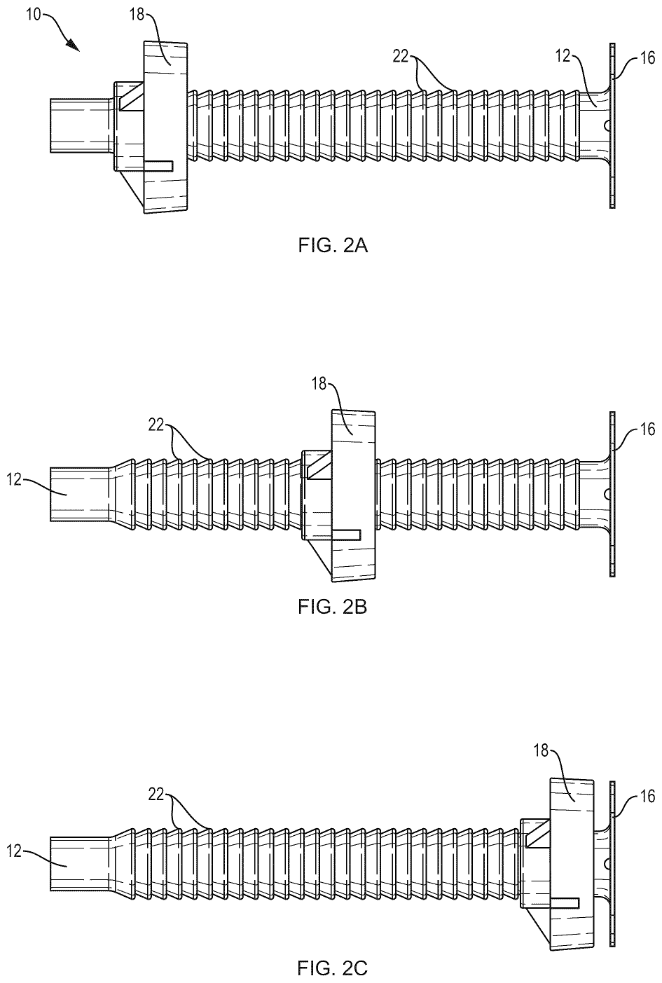

FIGS. 2A-2C illustrate the movement of the moveable member 18 along a length of the body 12. As shown in FIGS. 2A-2C, at least one of the body 12 (including the first engagement features 22) and the moveable member 18 may comprise a flexible material, such as silicone rubber, that allow the engagement features 22, 26 to flex and bend to allow movement of the moveable member 18 along a length of the body 12.

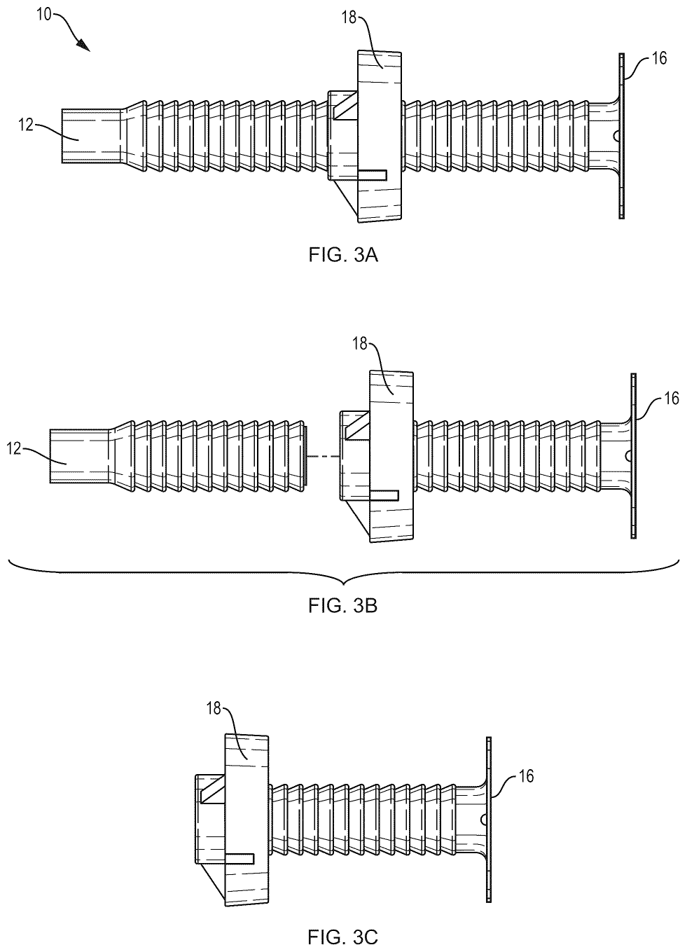

FIGS. 3A-3C illustrate the use of the cannula 10 in an arthroscopic surgical repair. As shown in FIG. 3A, when the cannula 10 is inserted into the joint space of a patient, the surgeon can place the flange 16 into contact with an inside tissue of the patient such that it is supported by the inside tissue. The surgeon can then adjust the moveable member 18 to rest against the outside skin on the patient. As shown in FIG. 3B, once the moveable member 18 has been moved to the desired position, the surgeon can trim the portion of the body 12 proximal to the moveable member 18. As shown in FIG. 3C, the surgeon can then use the trimmed cannula 10 to pass surgical instruments, sutures, and grafts during the repair. The trimmed cannula 10 thus may allow both improved visualization of the repair and increased stabilization of the cannula 10 within the patient's body.

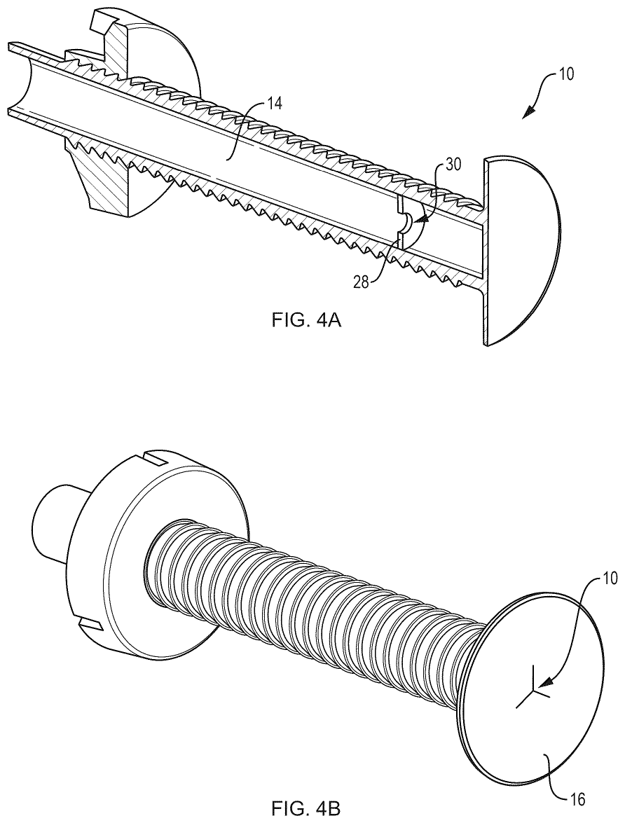

FIGS. 4A and 4B illustrate features of the cannula 10 that allow the surgeon to prevent back-flow of fluid from the patient's body. As shown in FIG. 4A, the through hole 14 of the cannula 10 may include an internal dam 28 positioned transverse to a longitudinal axis of the through hole 14. The dam 28 may define an opening 30 having a diameter selected to be smaller than the diameter of the through hole 14. A size of the opening 30 may be selected to allow passage of surgical instruments while simultaneously forming a seal around the instrument. The seal may prevent back-flow of fluid through the cannula 10 when the surgeon passes the instrument through the cannula 10. In addition, as shown in FIG. 4B, the flange 16 may include a slit feature 32 formed through a width of the flange 16. The slit feature 32 may be in the form of a “Y” cut that may allow for passage and removal of instruments through the “Y” cut, while also sealing the cannula 10 off from fluids when no instrument is present. However, the disclosure contemplates other configurations of the slit feature 32.

FIGS. 5A-5C illustrate features of the flexible flange 16 that allow for bending of the flange 16 during insertion of the cannula 10 into the patient's body. As shown in FIG. 5A, a cross section of the flange 16 may comprise a webbing pattern such that webbed areas 34 of the flange 16 are thinner than surrounding areas of the flange 16. The thinner webbed areas 34 may allow the flange 16 to bend along the webbed areas 34 to allow for easier insertion into and removal of the cannula 10 from the patient's body. In other embodiments, shown in FIG. 5B, opposing webbed areas 34 may additionally include a hole 36 to allow for engagement with a portion of an insertion instrument, which may hold the flange 16 in the bent position as it is being inserted into the patient's body. Furthermore, while a shape of the flange 16 may be circular as shown in FIGS. 5A and 5B, a shape of the flange 16 could also be oblong or oval as shown in FIG. 5C. The oblong or oval shape of the flange 16 may allow for a reduction of the footprint of the flange 16 during insertion into the patient's body. The disclosure also contemplates other suitable shapes of the flange 16, such as pear-shaped and other asymmetrical shapes.

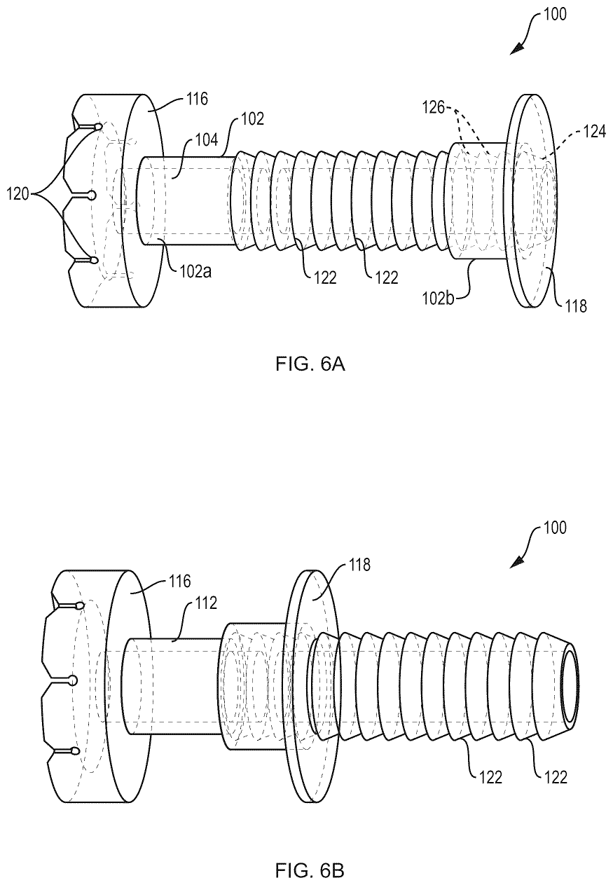

FIGS. 6A and 6B illustrate an alternative embodiment of an adjustable flexible cannula 100 of this disclosure. As shown in FIG. 6A, the cannula 100 may generally include a tubular body 102 having a through hole 104 extending from a proximal end 102a to a distal end 102b of the body 102. An outer surface of the body 102 may include a plurality of axially spaced first engagement features 122 extending around a circumference of the body 102. A flange 116 may be provided at the proximal end 102a of the body 102. The flange 116 may include a plurality of suture management features 120 for holding tensioned sutures in place as instruments and grafts are passed through the cannula 100. A moveable member 118, which may also be a flange, may be disposed about the main body 102 towards the distal end 102b of the body 102. The moveable member 118 may define a lumen 124 for receiving the body 102 therethrough. An inner surface of the lumen 124 may comprise a plurality of second engagement features 126 for selectively engaging a corresponding number of first engagement features 122 to tighten the interface between the moveable member 118 and the body 102. For example, the first engagement features 122 may be cylindrical barbs having a negative rake angle such that the face of the barb slopes away from the outer edge of the barb. The second engagement features 126 may in turn be a selected number of cylindrical channels, each one configured to receive a corresponding one of the barbs.

As shown in FIG. 6B, at least one of the body 112 (including the first engagement features 122) as well as the moveable member 118 may comprise a flexible material, such as silicone rubber, that allow the engagement features 122, 126 to flex and bend to allow movement of the moveable member 118 along a length of the body 112. Thus, the moveable member 118 may allow a length of the cannula 100 between the outside skin and the inside tissue to be adjusted based on the individual characteristics of the patient being treated. Once the moveable member 118 has been moved to the desired position, the surgeon can trim the portion of the body 112 distal to the moveable member 118 before passing instruments or grafts through the cannula 100.

FIGS. 7A and 7B illustrate another embodiment of an adjustable flexible cannula 200 of this disclosure. The cannula 200 may be substantially the same as cannula 10 except as described below. As shown in FIG. 7A, the cannula 200 may generally include a tubular body 212 having a through hole 214 extending from a proximal end 212a of the body 212 to a distal end 212b. A flexible flange 216 may be provided at the distal end 212b of the body 212. A moveable member 218, which may also be a flange, may be disposed about the main body 212 towards the proximal end 212a of the body 212. As shown in FIG. 7B, an inner surface of the moveable member 218 may comprise a plurality of second engagement features 226 for selectively engaging a corresponding number of first engagement features 222 on an outer surface of the body 212. The cannula 200 may also include a slit feature 232 formed through a width of the body 212 and located proximally of the flange 216. The slit feature 232 may be in the form of a “Y” cut that may allow for passage and removal of instruments through the “Y” cut, while also sealing the cannula 200 off from fluids when no instrument is present. However, the disclosure contemplates other configurations of the slit feature 232.

While the disclosure particularly shows and describes preferred embodiments, those skilled in the art will understand that various changes in form and details may exist without departing from the spirit and scope of the present application as defined by the appended claims. The scope of this present application intends to cover such variations. As such, the foregoing description of embodiments of the present application does not intend to limit the full scope conveyed by the appended claims.

1. A cannula comprising:
a body having a proximal end and a distal end, the body defining a through hole extending from the proximal end to the distal end, the distal end of the body comprising a flange for engaging a surface of tissue, an outer surface of the body comprising a plurality of axially spaced first engagement features extending around a circumference of the body; and
a moveable member having a lumen for receiving the body therethrough, an inner surface of the lumen comprising a plurality of second engagement features for selectively engaging a corresponding one of the first engagement features;
wherein the moveable member is moveable along a length of the body to adjust a distance between the flange and the moveable member.

2. The cannula of claim 1, wherein the moveable member is a flange.

3. The cannula of claim 1, wherein the moveable member comprises a plurality of suture management features.

4. The cannula of claim 1, wherein the first engagement features are cylindrical barbs having a negative rake angle such that the face of the barb slopes away from the outer edge of the barb.

5. The cannula of claim 4, wherein the second engagement features are cylindrical channels, each channel configured to receive a corresponding one of the barbs.

6. The cannula of claim 1, wherein the plurality of second engagement features is at least three second engagement features.

7. The cannula of claim 1, wherein the plurality of second engagement features is five second engagement features.

8. The cannula of claim 1, wherein at least one of the body and the moveable member comprise a flexible material.

9. The cannula of claim 1, wherein the through hole includes an internal dam defining an opening therethrough, a diameter of the opening selected to be smaller than a diameter of the through hole.

10. The cannula of claim 9, wherein a size of the opening is selected to allow passage of a surgical instrument while simultaneously forming a seal around the surgical instrument.

11. The cannula of claim 1, further comprising a slit feature configured to allow for passage and removal of a surgical instrument while simultaneously forming a seal around the surgical instrument.

12. The cannula of claim 11, wherein the slit feature is located proximal of the flange.

13. The cannula of claim 1, wherein a cross section of the flange comprises a plurality of webbed areas, a width of the webbed areas selected to be thinner than a width of a surrounding area of the flange to allow the flange to bend along the webbed areas.

14. The cannula of claim 13, wherein opposing webbed areas of the flange define a hole therethrough, the hole configured to engage an insertion instrument passed through the cannula.

15. A method of inserting a cannula into a body of a patient, the method comprising:
placing a flange of the cannula into contact with an internal tissue the patient, the cannula further comprising:
a body having a proximal end and a distal end, the body defining a through hole extending from the proximal end to the distal end, the distal end of the body comprising the flange, an outer surface of the body comprising a plurality of axially spaced first engagement features extending around a circumference of the body; and
a moveable member having a lumen for receiving the body therethrough, an inner surface of the lumen comprising a plurality of second engagement features for selectively engaging a corresponding one of the first engagement features; and
moving the moveable member along a length of the body to adjust a distance between the flange and the moveable member.

16. The method of claim 15, wherein moving the moveable member comprises placing the moveable member into contact with an outside skin of the patient.

17. The method of claim 15, further comprising trimming the body of the cannula distal to the moveable member.

18. A method of inserting a surgical instrument into a body of a patient, the method comprising:
placing a flange of the cannula into contact with an internal tissue the patient, the cannula further comprising:
a body having a proximal end and a distal end, the body defining a through hole extending from the proximal end to the distal end, the distal end of the body comprising the flange, an outer surface of the body comprising a plurality of axially spaced first engagement features extending around a circumference of the body; and
a moveable member having a lumen for receiving the body therethrough, an inner surface of the lumen comprising a plurality of second engagement features for selectively engaging a corresponding one of the first engagement features; and
moving the moveable member along a length of the body to adjust a distance between the flange and the moveable member; and
passing the surgical instrument through the cannula into the body of the patient.

19. The method of claim 18, further comprising passing the surgical instrument through an opening of an internal dam of the cannula, a diameter of the opening selected to be smaller than a diameter of the through hole.

20. The method of claim 18, further comprising passing the surgical instrument through a slit feature of the cannula, the slit feature configured to allow for passage and removal of the surgical instrument while simultaneously forming a seal around the surgical instrument.
